(19) United States
(12) Patent Application Publication
(10) Pub. No.: US 2005/0272692 A1
(43) Pub. Date: Dec. 8, 2005

(54) Sucrose ester compounds useful as fungicides

(76) Inventors: William A. Farone, Irvine, CA (US); Tracy Palmer, Fallbrook, CA (US)

Correspondence Address: Cynthia H. O'Donohue;Applied Power Concepts, Inc., 411 East Julianna Street, Anaheim, CA, 92801, US

(21) Appl. No.: 11/144,988

(22) Filed: Jun. 3, 2005

Related U.S. Application Data
(60) Provisional application No. 60/577,290, filed on Jun. 4, 2004.

(51) Int. Cl. A61K 31/7024

(52) U.S. Cl. 514/53

(57) ABSTRACT

The preparation of sucrose esters of Formula I (RZnX) are described and their use as environmentally friendly antifungal compounds where R is 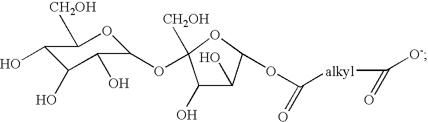 and X is selected from the group consisting of halogen, hydroxyl, and R.

This application claims the benefit of U.S. Provisional Application No. 60/577,290, filed Jun. 6, 2004.

### FIELD OF THE INVENTION

The present invention relates to the preparation of and the use of sucrose esters which are environmentally friendly and are useful as antifungal compounds.

### BACKGROUND OF THE INVENTION

The following description of the background of the invention is provided to aid in understanding the invention, but is not admitted to be, or to describe, prior art to the invention. All publications are incorporated by reference in their entirety.

Recent studies on sucrose esters have shown them to be useful as environmentally friendly and safe insecticides. In these studies the sucrose esters were found to be well tolerated by man and extremely biodegradable

In the 1970s a series of cosmetic and detergent formulations were developed based on sucrose esters. U.S. Pat. No. 4,151,304 (Method and Composition for Moisturizing the Skin) described the succinate half esters of sucrose that were found to make excellent products for human skin applications. Sodium and potassium salts were used in these applications. This work showed these compounds to be considered mild and well tolerated by man.

Poller and Parkin (“Organometallic Derivatives of Sucrose as Pesticides”” in the book “Sucrochemistry” (edited by John L. Hickson and published by The American Chemical Society in 1977, ACS Symposium Series 41) reported on the use of triphenyllead sucrose succinate, tributyltin sucrose succinate and triphenyltin sucrose succinate as pesticides. The use of lead and tin is not desirable today and furthermore it is not desirable to make the phenyl esters since breakdown compounds from the phenyl esters are toxic products like phenol. Therefore the use of such compounds would add to the environment additional toxic pollutants. Thus there exists a need to have environmentally friendly compounds that are specific to either killing a wide range of fungi or preventing the growth of the fungi.

### DEFINITIONS

In accordance with the present invention and as used herein, the following terms are defined with the following meanings, unless explicitly stated otherwise.

The term “enhanced” refers to increasing or improving a specific property. The term “effective amount” refers to an amount that decreases or inhibits the growth of a fungus by 50%.

The term ‘alkyl’ refers to a straight or branched carbon chain of two to six carbons. The term ‘carrier’ refers to a means of transmitting active substance. Typically it is a neutral substance to which an active ingredient or agent is added as a way of applying or transferring the ingredient or agent. In one aspect the carrier is water.

The following abbreviations are used: TLC: thin layer chromatography ppm: parts per million

### SUMMARY OF THE INVENTION

The present invention is directed towards a method of inhibiting the growth of fungi in which the fungi are treated with a compound of Formula I
RZnX
wherein:
R is 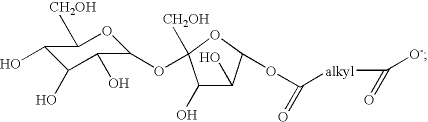
X is selected from the group consisting of halogen, hydroxyl, and R;
   and alkyl is C₂-C₆. In one aspect the fungi are independently selected from the group consisting of Pythium ultimum, Rhizoctonia solani, Fusarium graminearum, Alternaria sp., Stachybotrys chartarum, Aspergillus niger, and Muco sp. In a further aspect the fungi are treated with a composition containing an effective amount of the compound of Formula I in an environmentally acceptable carrier. In an additional aspect this carrier is water.

This instant invention in an additional aspect is directed towards a method for killing fungi where the fungi are treated with a compound of Formula I
RZnX
wherein:
R is 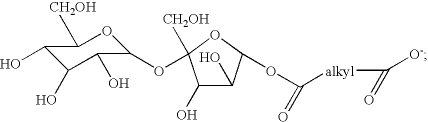
X is selected from the group consisting of halogen, hydroxyl, and R;
   and alkyl is C₂-C₆. In one aspect the fungi are independently selected from the group consisting of Pythium ultimum, Rhizoctonia solani, Fusarium graminearum, Alternaria sp., Stachybotrys chartarum, Aspergillus niger, and Muco sp. In a further aspect the fungi are treated with a composition containing an effective amount of the compound of Formula I in an environmentally acceptable carrier. In an additional aspect this carrier is water.

In a further aspect this present invention is directed towards a method of inhibiting the growth of fungi in which the fungi are treated with a compound of Formula II
R₂Zn
wherein:
R is 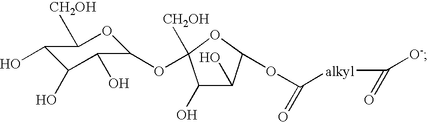
   and alkyl is C₂-C₆. In one aspect the fungi are independently selected from the group consisting of Pythium ultimum, Rhizoctonia solani, Fusarium graminearum, Alternaria sp., Stachybotrys chartarum, Aspergillus niger, and Muco sp. In a further aspect the fungi are treated with a composition containing an effective amount of the compound of Formula II in an environmentally acceptable carrier. In an additional aspect this carrier is water.

An additional aspect is directed towards a method for killing fungi where the fungi are treated with a compound of Formula II
R₂Zn
wherein:
R is 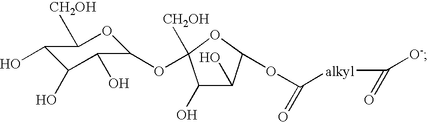
   and alkyl is C₂-C₆. In one aspect the fungi are independently selected from the group consisting of Pythium ultimum, Rhizoctonia solani, Fusarium graminearum, Alternaria sp., Stachybotrys chartarum, Aspergillus niger, and Muco sp. In a further aspect the fungi are treated with a composition containing an effective amount of the compound of Formula II in an environmentally acceptable carrier. In an additional aspect this carrier is water.

This instant invention is also directed to a method of making compound of Formula I:
RZnX
wherein:
R is 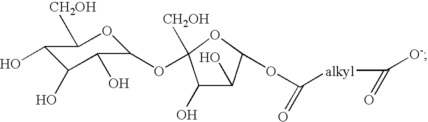
   X is selected from the group consisting of halogen and hydroxyl; and the alkyl is C₂ to C₆ and to a method of making this compound of Formula I in a safe and environmentally friendly manner. Sucrose and a C₂ to C₆ alkyl carboxylic acid anhydride are combined and heated. An aqueous solution of a zinc salt is added, followed by neutralizing with recovery of the desired compound from the aqueous solution. In one aspect the temperature used for heating is from 110 to 140° C. In another aspect the zinc salt is ZnCl₂. The neutralizing agent in an additional aspect is sodium hydroxide.

A further aspect is the synthesis of a compound of Formula II:
R₂Zn
wherein:
R is 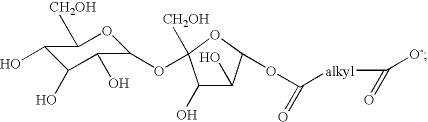
   and alkyl is C₂-C₆ and to a method of making this compound of Formula II in a safe and environmentally friendly manner. Sucrose and a C₂ to C₆ alkyl carboxylic acid anhydride are combined and heated. An aqueous solution of a zinc salt is added, followed by neutralizing with recovery of the desired compound from the aqueous solution. In one aspect the temperature used for heating is from 110 to 140° C. In another aspect the zinc salt is ZnCl₂. The neutralizing agent in a further aspect is sodium hydroxide.

An additional aspect is directed towards a method of inhibiting the growth of fungi by treating the fungi with an effective amount of a compound selected from the group consisting of sucrose octanoate and sorbitol octanoate. In one aspect these fungi are selected from the group consisting of Pythium ultimum, Rhizoctonia solani, Fusarium graminearum, Alternaria sp., Stachybotrys chartarum, Penicillium sp., Rhizopus spp., and Muco sp

### BRIEF DESCRIPTION OF THE DRAWINGS

FIG. 1 Fungus colony size in cm of Pythium ultimum in hours with concentrations of Compound A from 10 to 5000 ppm.

FIG. 2 Fungus colony size in cm of Rhizoctonia solani in hours with concentrations of Compound A from 10 to 5000 ppm.

FIG. 3 Fungus colony size in cm of Mucor sp. In hours with concentrations of Compound A from 10 to 5000 ppm.

FIG. 4 Fungus colony size in cm of Alternaria sp. in hours with concentrations of Compound A from 10 to 5000 ppm.

FIG. 5 Fungus colony size in cm of Fusarium graminearum. in hours with concentrations of Compound A from 10 to 5000 ppm.

FIG. 6 Fungus colony size in cm of Strachybotrys chartarum in hours with concentrations of Compound A from 1000 to 5000 ppm.

FIG. 7 Fungus colony size in cm of Aspergillus niger in hours with concentrations of Compound A from 1000 to 5000 ppm.

FIG. 8 Radial growth in mm of Rhizopus stolonifer in hours with concentrations of sucrose octanoate from 400 to 2000 ppm.

FIG. 9 Radial growth in mm of Stachyboterys chartarum in hours with concentrations of sucrose octanoate from 400 to 2000 ppm.

FIG. 10 Radial growth in mm of Penicillium chrysogenum in hours with concentrations of sucrose octanoate from 400 to 2000 ppm.

FIG. 11 Radial growth in mm of Rhizopus stolonifer in hours with concentrations of sucrose octanoate from 400 to 2000 ppm.

FIG. 12 Radial growth in mm of Stachyboterys chartarum in hours with concentrations of sorbitol octanoate from 400 to 2000 ppm.

FIG. 13 Radial growth in mm of Aspergillus niger in hours with concentrations of sorbitol octanoate from 400 to 2000 ppm.

FIG. 14 Radial growth in mm of Penicillium chrysogenum in hours with concentrations of sorbitol octanoate from 400 to 2000 ppm.

### DETAILED DESCRIPTION OF THE INVENTION

The structure of a simple sucrose succinate molecule is shown in Formula Ia: 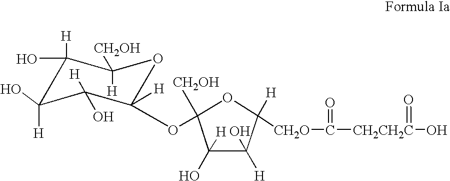
For ease and convenience in drawing, the succinate is drawn on the right hand side of the sucrose molecule using an available hydroxyl group. However, the succinate moiety can be located on any of the eight OH groups of the sucrose molecule. Using molecular orbital modeling it has been shown with stearate esters that only one of these positions is less favored than the other seven. This is supported by the fact that 6-7 bands identified with the mono esters are seen when the stearate or other fatty acid esters are examined by thin layer chromatography (TLC). There is normally clear separation between the mono and higher esters when examined by TLC.

In this instant invention of an antifungal compound the free end of the succinate group was replaced by zinc ion. Zinc is known to be reasonable safe to man and has been used in zinc oxide and zinc undecylenate for antiseptic and antifungal purposes. These materials are applied locally in large amounts as either powders or creams.

Zinc usually stimulates enzymatic processes in fungi so that the fungi can digest materials needed for metabolism. Thus it was unexpected and surprising that zinc salts of a mild, skin safe anion inhibited the growth of the fungi when applied from aqueous systems at very low concentrations.

Apparently, in retrospect, the activity of the zinc is coupled with the cell affinity of the sucrose succinate molecule. The resultant compound was unexpectedly found to work equally on most types of surfaces including plant tissues since sucrose esters are naturally found in leaves and a related compound, sucrose octanoate, is known to be useful as a foliar insecticide. Sucrose octanoate and the related sorbitol octanoate synthesis and their use as insecticides were described in U.S. Pat. No. 6,419,941 incorporated herein by reference. The inventors describe in '941 the result that octanoic acid (C8) sorbitol esters were more effective as insecticides than other members of the class of polyol fatty acid esters of significantly larger or smaller size. There was no mechanistic or theoretical way to determine which size molecule or of what range of substituents had the insecticidal or fungicidal effect other than by direct testing separately for each activity.

Also there was an unexpected finding in '941 that for sucrose octanoate the monoesters were more effective as insecticides than the diesters and triesters. Their finding was in contradiction to earlier work of Chortyk (J. Agric. Food Chem, 44, 1551-7 (1996)). Additionally it was found that a mixture of sorbitol octanoate and sucrose octanoate had a greater insecticide activity than was expected from an additive effect (U.S. Pat. No.6,756,046 B2).

These two octanoates were found to have activity against some of the fungi. This finding that these octanoates were selective and the zinc salts of sucrose esters had a broad spectrum of activity was surprising. There are no predictive tools that can describe this difference. It was only by testing and the availability of the octanoate materials from the previous work that the results of the current invention could be determined. The potential commercial uses of the fungicides can be arranged to target all fungi or selected classes of fungi. For example, in some instances where certain fungi are desired (commercial biochemical transformations), other competing fungi can be repressed while maintain the desired population. The octanoate esters are not effective on Aspergillus. Aspergillus are used in the fermentation of sugars to produce citric acid. Thus the octanoate compounds would be useful in sugar fermentation to preserve the Aspergillus cultures from invasion by other fungi.

The synthesis of the desired zinc sucrose succinate compound was found to be economically viable. It is known to those skilled in the art, that sucrose succinate can be made directly but incompletely from sucrose and succinic anhydride with limited or no by-products. This is usually accomplished by using solvents, such as pyridine. Solvents such as pyridine are not considered to be environmentally friendly. The Occupational Safety and Health Administration (OSHA) has set an occupational exposure limit of 5 parts of pyridine per million parts of workplace air (5 ppm) for an 8-hour workday over a 40-hour workweek. This and similar solvents have to be removed prior to making the desired salts from aqueous solutions. In this instant invention a method was found that allowed the synthesis to occur without the use of such potential hazardous solvents. Removal of this step made the synthesis economically viable in addition to being more environmentally friendly.

Sucrose succinate was made directly from sucrose and succinic anhydride with no by-products and was then neutralized. The only other material used was water with a base to neutralize the chloride from the zinc salt, which is removed at the end of the manufacturing process. It was found that the amount of zinc chloride added in the water was equal to approximately 15 per cent zinc concentration to the sucrose succinate.

The zinc coupled with the sucrose succinate and the chloride coupled with the base used for neutralization. Sodium chloride and zinc sucrose succinate were formed. The desired zinc sucrose succinate precipitated from the reaction solution. The sodium chloride stayed in the water but could be removed by evaporation if so desired.

A representative structure for the compound, zinc mono-sucrose succinate, is shown in Formula Ib: 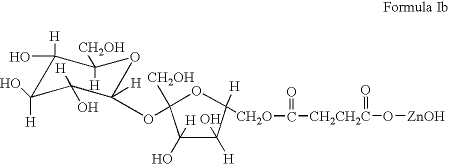
The di-sucrose succinate salt may also form:
ZnR₂

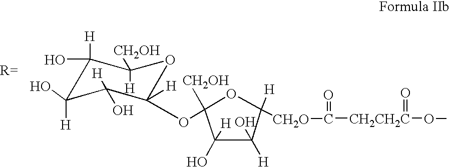

The compounds used in this invention, their preparation, and their use can be understood further by the examples which illustrate some of the processes by which these compounds are prepared. These examples should not however be construed as specifically limiting the invention and variations of the compounds, now known or later developed, are considered to fall within the scope of the present invention as hereinafter claimed.

### EXAMPLES

### Example 1

### Synthesis of Zinc Mono-Sucrose Succinate: Compound A

Compound A was synthesized by the following procedures.

### Example 1A

A one to one mole ratio of succinate anhydride and sucrose (10.02 grams of succinate anhydride and 34.21 grams of sucrose) were added to a 250 mL beaker with a stir bar and the contents was heated to 140° C. The solution was an amber color. Water was removed through vaporization and the solution became very thick. After 45 minutes there was no more visual water being removed. The beaker was cooled to room temperature. A 100 ml of a 20% zinc chloride was made (9.59 grams of zinc or 0.147 moles of zinc). The Zn solution was added to the warm sucrose succinate with a pH of 1.5. The resulting solution was neutralized with a 43% solution of sodium hydroxide. When the pH reached 5.0, precipitates starting forming. Neutralization was continued until the solution has a pH of 7.0. The precipitated product (A) was filtered and the solids were washed three times with water to remove any excess salt. Compound A was placed in a vacuum oven (40° C., Hg 29.5 ins.) to remove any excess water.

Compound A color: orange powder

Solubility: not soluble in water

### Example 1B

Sucrose (35.26 grams) was added to a 400 ml beaker. The beaker was placed into a mineral oil bath on a hot plate. The sucrose was heated slowly. When the temperature was around 80° C., 10.25 grams of succinate anhydride was added to the beaker. The reaction was stirred. When the temperature in the mineral oil bath reached 135° C., the heat was turned off. The reaction temperature was about 120° C. The mixture was a soft gel and the color was yellow. The reaction temperature climbed to 135° C. The mixture became a very thick liquid. The beaker was removed from the heat and placed into an ice bath. A zinc chloride solution (68 grams of zinc chloride in 100 grams water) was added until the pH was 0.2. The mixture was neutralized with a 20% sodium hydroxide solution. When the pH reached 5.0, precipitates were formed. The product was neutralized until a pH of 7.0 was obtained. The solution was filtered using a vacuum filtration apparatus. The solids were washed three times with distilled water to remove any excess salt. The product was placed into a vacuum dryer at 29 inches of mercury. The dried solids were an orange color. The product was ground into a fine powder.

### Example 2

### Efficiency of Compound A

### Example 2A

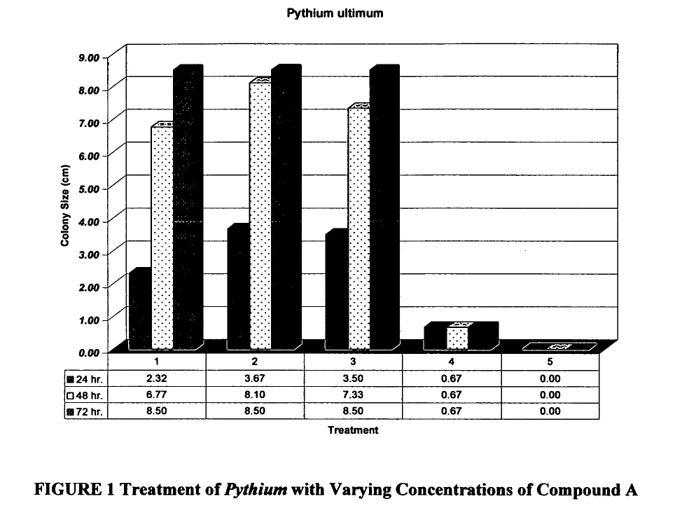

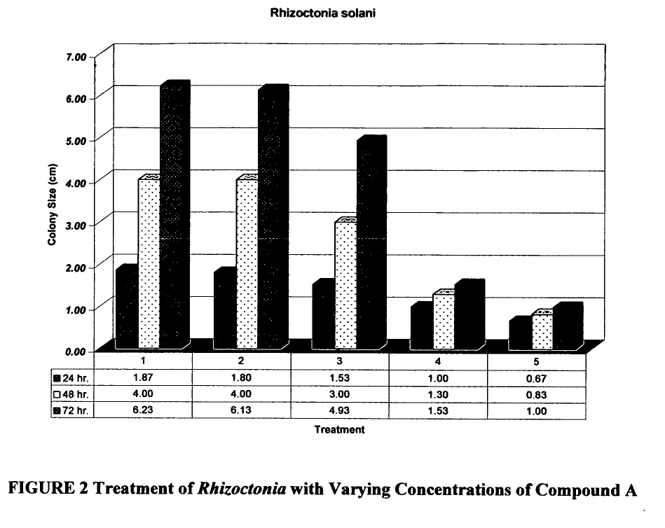

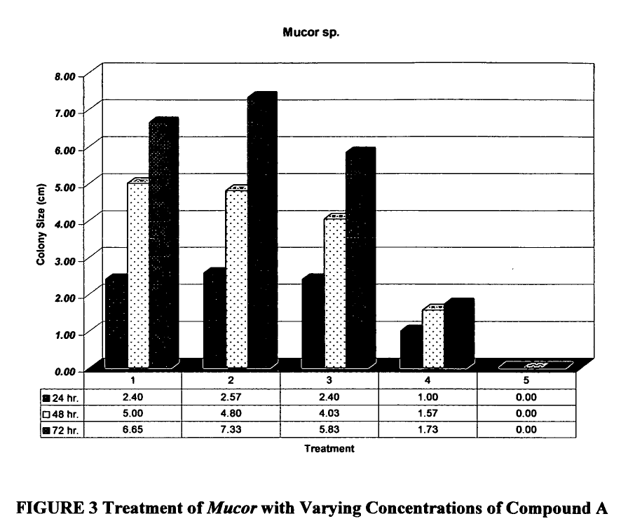

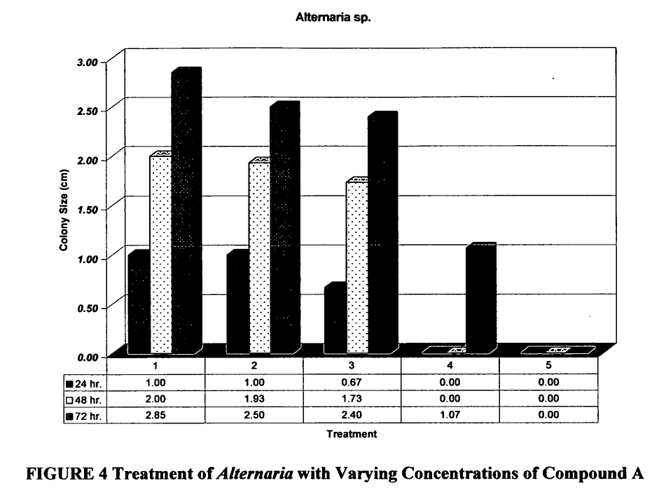

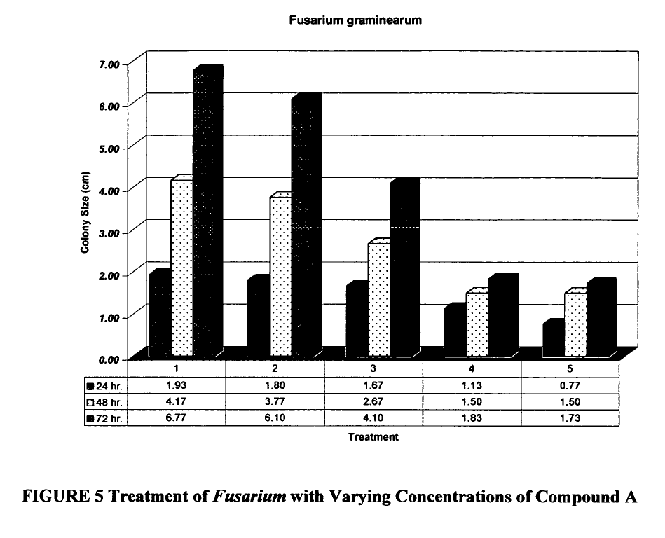

Compound A was tested against several fungi by the Gustafson Seed Technology Center, McKinney, Tex. The compound was tested at levels of 10, 100, 1000 and 5000 ppm against Pythium ultimum, Rhizoctonia solani, Fusarium graminearum, Alternaria sp. and Muco sp. The results are shown in FIGS. 1 to 5. The treatment values are 1=control, 2=10 ppm, 3=100 ppm, 4=1000 ppm and 5=5000 ppm.
Pythium ultimum: At 1000-ppm, radial growth was reduced from an 85 mm diameter to a 6.6 mm, 92.3% reduction. At 5000-ppm, no significant level of growth was observed.
Rhizoctonia solani: At 1000-ppm, radial growth was reduced from 62.3 mm diameter to 15.3 mm, 75.4% reduction. There was very limited growth at 5000-ppm with a measurement of 10 mm recorded.
Fusarium graminearum: At 1000-ppm, radial growth was reduced from 67.6 mm diameter to 18.3 mm, 73% reduction. The assay at the 5000-ppm showed no additional inhibition over the 1000-ppm assay.
Alternaria sp: At 1000-ppm, radial growth was reduced to 10 mm diameter from 28.5 mm, 64.9% reduction. At 5000-ppm, no significant level of growth was observed.
Mucor sp: At 1000-ppm, radial growth was reduced from 66.5 mm diameter to 17 mm, 74.4% reduction. At 5000-ppm, no significant level of growth was observed.

### Example 2B

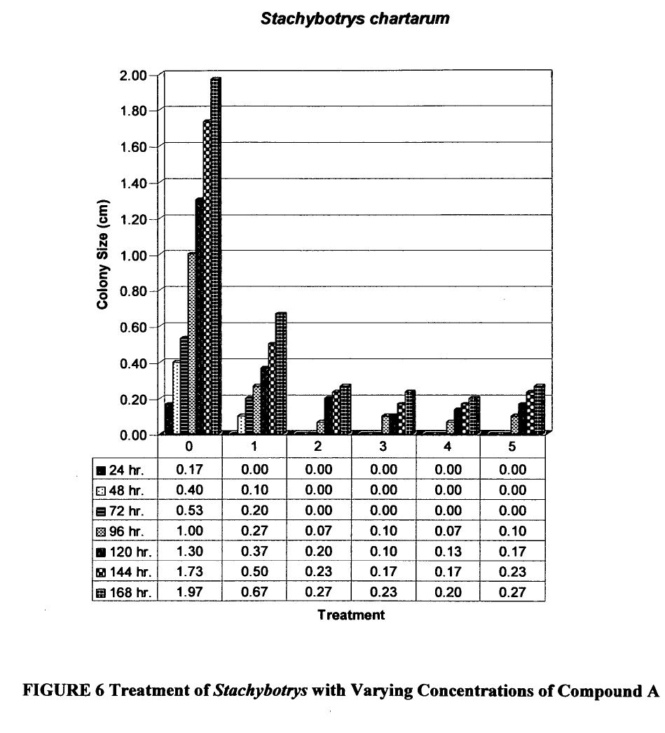

Stachybotrys chartarum: At 1000-ppm, radial growth was reduced from a 19.7 mm diameter on the control medium to 6.7 mm, 66.0% reduction. Sporulation was reduced to low levels on the 1000-ppm medium, compared to high levels on the control medium. There was very limited growth at 5000-ppm with a measurement of 2.7 mm recorded. FIG. 6 shows the effects of different concentrations of Compound A where 1 is 1000 ppm, 2 is 2000 ppm, 3 is 3000 ppm, 4 is 4000 ppm, and 5 is 5000 ppm. The control (no treatment) is identified by 0.

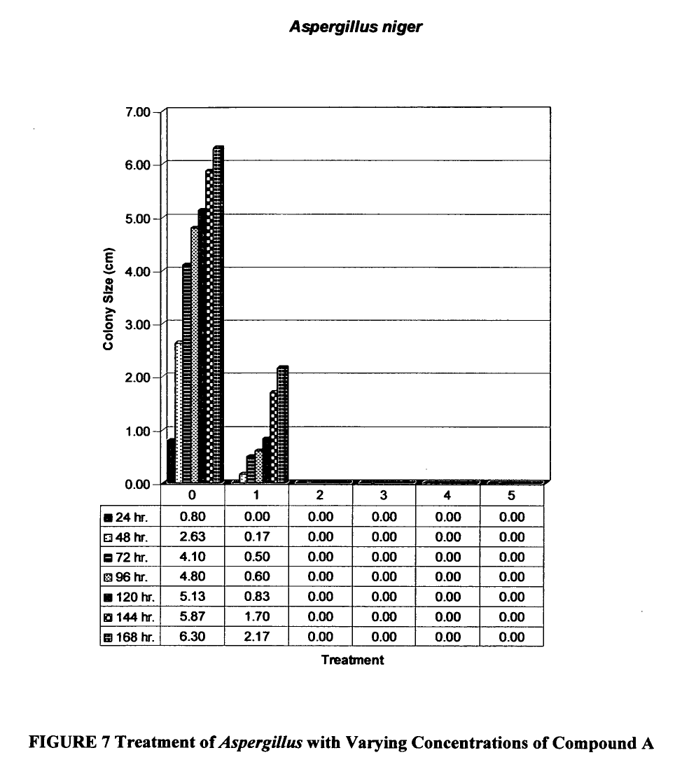

Aspergillus niger: At 1000-ppm, radial growth was reduced from a 63.0 mm diameter on the control medium to 21.7 mm, a 65.6% reduction. Sporulation was high on both the 1000-ppm medium and the control medium. At 5000-ppm, no significant level of growth was observed. FIG. 7 shows the effects of different concentrations of Compound A where 1 is 1000 ppm and 5 is 5000 ppm. The control (no treatment) is identified by 0.

### Example 3

### Efficiency of Sucrose Octanoate

Sucrose octanoate at 2000 ppm prevented the growth of three different fungi in a lab trial. At 1000 ppm sucrose octanoate prevented the growth of Stachybotrys and Rhizopus. When this compound was used for Penicillium at 1000 ppm there was some growth of the fungus.

### Example 4

### Efficiency of Sucrose Octanoate against Five Indoor Fungi

Five common indoor air fungi were tested against sucrose octanoate to determine if the compound had antifungal activity. For each fungus tests were conducted on malt extract agar that was amended with five concentrations of sucrose octanoate. The control medium was standard malt extract agar. The concentrations were prepared based on 100% active ingredient. The parts per million were calculated afterwards to account for a concentration of 40% active ingredient. All fungi in the tests were incubated at 25° C. for 144 hours.

### Example 4A

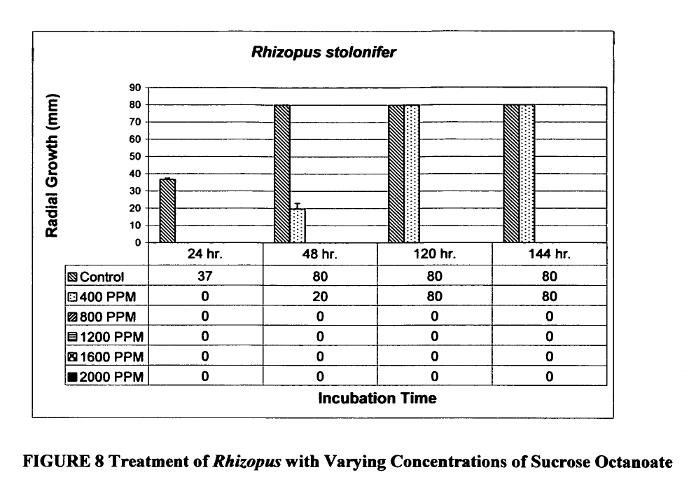

Growth of Rhizopus stolonifer, the common bread mold, was inhibited at all concentrations of sucrose octanoate tested. (FIG. 8). There was no growth during the 144 hour incubation period with the exception of sucrose octanoate at 400 ppm level. Rhizopus is a Zygomycetazoan,

### Example 4B

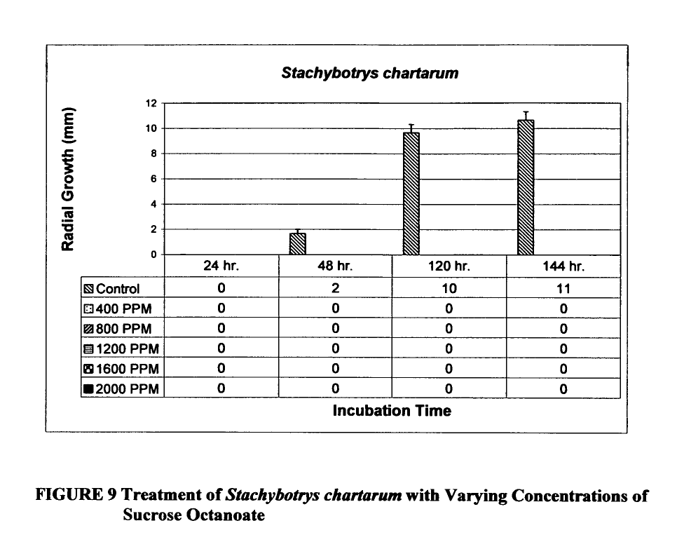

Growth of Stachybotrys chartarum, toxic black mold, was inhibited at all concentrations of sucrose octanoate (FIG. 9). No growth occurred during the 144 hour incubation period. S. chartarum, and the remaining fungi used in this experiment, are ascomycetozoans.

Ascomycetazoans make up 99% of the toxic and allergenic mold species found in indoor environments. They comprise the largest group of plant pathogenic fungi, and are responsible for destroying over a billion dollars worth of agriculture crops each year through direct yield loss, toxin production, and storage rot. Millions are spent applying expensive and often environmentally toxic fungicide each year in order to decrease the damage caused by plant pathogenic fungi.

### Example 4C

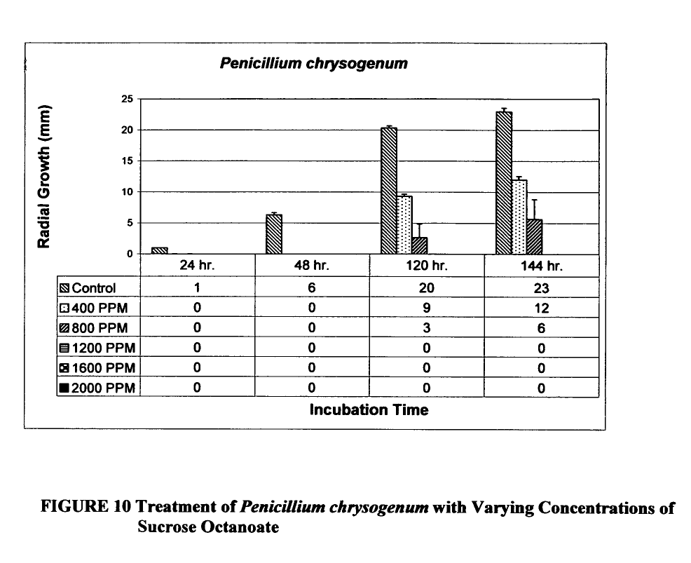

Penicillium chrysogenum was able to grow, although slowly compared to the control, at low concentrations of sucrose octanoate (FIG. 10). P. chrysogenum is a common toxin producing mold found in indoor environments and is often associated with water damaged materials.

### Example 4D

Aspergillus flavus and Aspergillus parasiticus were able to grow at all concentrations of sucrose octanoate. However, growth at 2000 ppm was significantly less (d.f=2, p<0.05) than growth on the control plates (see table), based on unpaired one-tailed T-tests. Both A. flavus and A. parasiticus are known human pathogens and toxin producers. A. parasiticus is responsible for lung infections and disease in immune-compromised patients.

### Example 5

### Efficiency of Sorbitol Octanoate

Sorbitol octanoate at 1000 ppm prevented the growth of both Stachybotrys and Rhizopus in a lab trial. When sorbitol octanoate (2000 ppm) was used for Penicillium, there was some growth of the fungus.

### Example 6

### Efficiency of Sorbitol Octanoate against Five Indoor Fungi

Five common indoor air fungi were tested against sorbitol octanoate to determine if the compound had antifungal activity. For each fungi tests were conducted on malt extract agar that was amended with five concentrations of sorbitol octanoate. The control medium was standard malt extract agar. The concentrations were prepared based on 100% active ingredient. The parts per million were calculated afterwards to account for a concentration of 40% active ingredient. All fungi in the tests were incubated at 25° C. for 144 hours.

### Example 6A

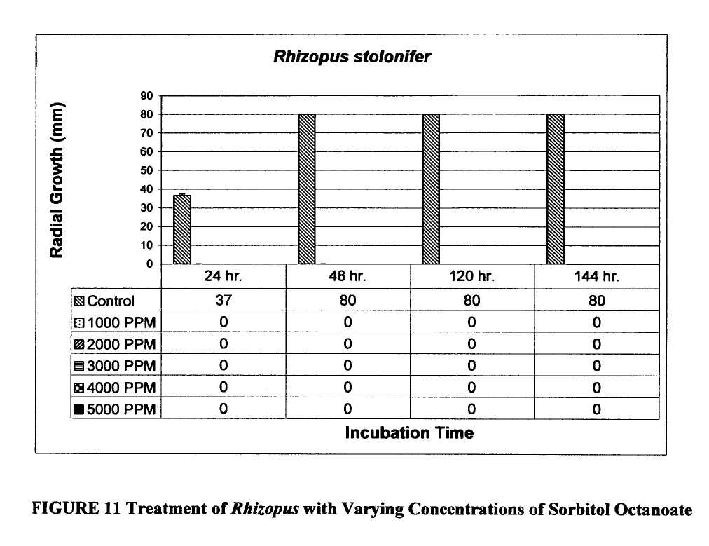

Growth of Rhizopus stolonifer, the common bread mold, was inhibited at all concentrations of sucrose octanoate tested. (FIG. 11). There was no growth during the 144 hour incubation period. Rhizopus is a Zygomycetazoan,

### Example 6B

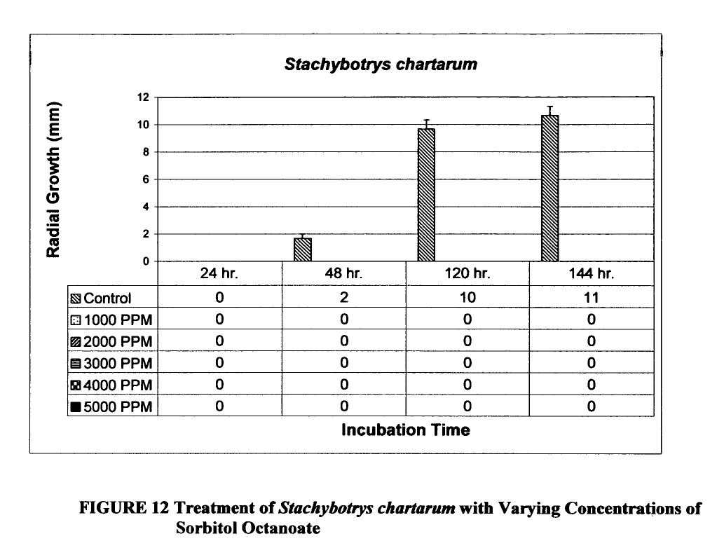

Growth of Stachybotrys chartarum, toxic black mold, was inhibited at all concentrations of sorbitol octanoate (FIG. 12). No growth occurred during the 144 hour incubation period. S. chartarum, and the remaining fungi used in this experiment, are ascomycetozoan.

Ascomycetazoans make up 99% of the toxic and allergenic mold species found in indoor environments. They comprise the largest group of plant pathogenic fungi, and are responsible for destroying over a billion dollars worth of agriculture crops each year through direct yield loss, toxin production, and storage rot. Millions are spent applying expensive and often environmentally toxic fungicide each year in order to decrease the damage caused by plant pathogenic fungi.

### Example 6C

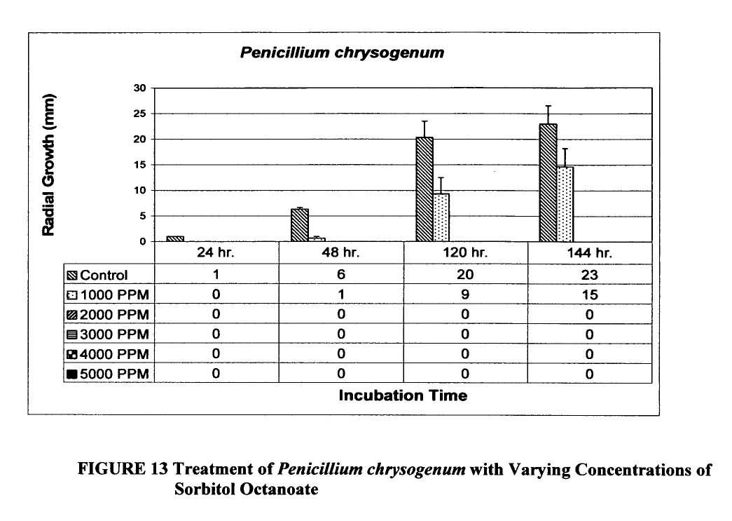

Penicillium chrysogenum was able to grow, although slowly compared to the control, at low concentrations of sorbitol octanoate (FIG. 13). P. chrysogenum is a common toxin producing mold found in indoor environments and is often associated with water damaged materials.

### Example 6D

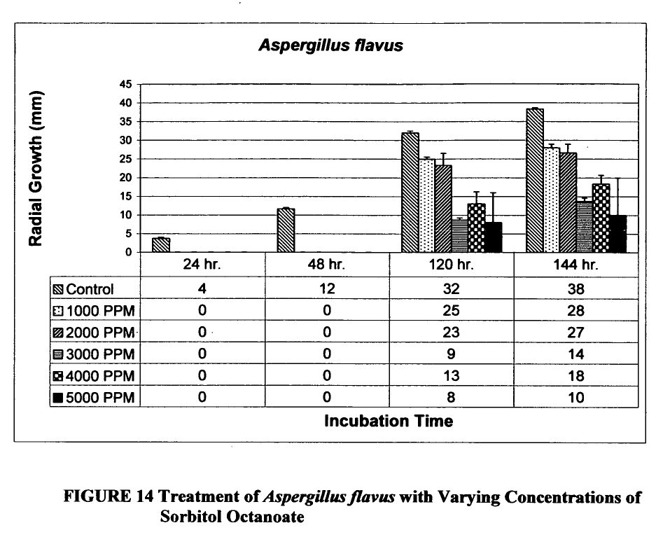

Aspergillus flavus and Aspergillus parasiticus were able to grow at all concentrations of sorbitol octanoate (FIG. 14). However, growth at 5000 ppm may be due to spurious data. Both A. flavus and A. parasiticus are known human pathogens and toxin producers. A. parasiticus is responsible for lung infections and disease in immune-compromised patients.

1. A method of inhibiting the growth of fungi comprising treating said fungi with a compound of Formula I:
RZnX
wherein:
R is 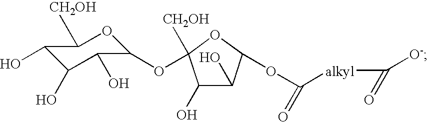
X is selected from the group consisting of halogen, hydroxyl, and R;
and alkyl is C₂-C₆.

2. The method of claim 1 wherein the fungi are independently selected from the group consisting of Pythium ultimum, Rhizoctonia solani, Fusarium graminearum, Alternaria sp., Stachybotrys chartarum, Aspergillus niger, and Muco sp.

3. A method of inhibiting the growth of fungi comprising treating said fungi with a composition comprising an effective amount of a compound of Formula I:
RZnX
wherein:
R is 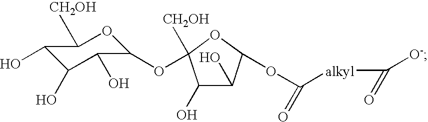
X is selected from the group consisting of halogen, hydroxyl, and R;
alkyl is C₂-C₆;
and an environmentally acceptable carrier.

4. A method for killing fungi comprising treating said fungi with a compound of Formula I:
RZnX
wherein:
R is 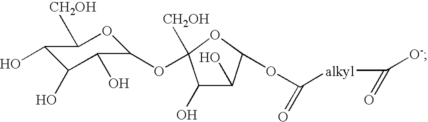
X is selected from the group consisting of halogen, hydroxyl, and R;
and alkyl is C₂-C₆.

5. The method of claim 4 wherein the fungi are independently selected from the group consisting of Pythium ultimum, Rhizoctonia solani, Fusarium graminearum, Alternaria sp., Stachybotrys chartarum, Aspergillus niger, and Muco sp.

6. A method for killing fungi comprising treating said fungi with a composition comprising an effective amount of a compound of Formula I:
RZnX
wherein:
R is 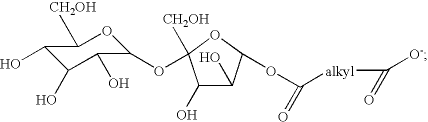
X is selected from the group consisting of halogen, hydroxyl, and R;
alkyl is C₂-C₆;
and an environmentally acceptable carrier.

7. A compound of Formula I:
RZnX
wherein:
R is 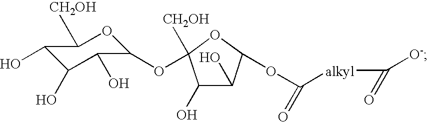
X is selected from the group consisting of halogen, hydroxyl, and R;
and alkyl is C₂-C₆.

8. A method of making compound of claim 8
wherein:
R is 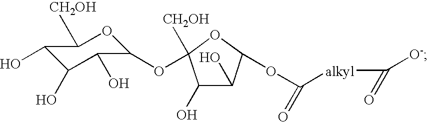
and alkyl is C₂-C₆;
comprising:
(a) combining sucrose and C₂-C₆ alkyl anhydride;
(b) heating the combination;
(c) adding an aqueous solution of zinc salt;
(d) neutralizing; and
(e) recovering compound from aqueous solution.

9. The method of claim 8 wherein the temperature for heating is 110° C. to 150° C.

10. The method of claim 9 wherein said zinc salt is ZnCl₂.

11. The method of claim 10 wherein said neutralizing agent is sodium hydroxide.

12. A method of inhibiting the growth of fungi comprising treating said fungi with an effective amount of a compound selected from the group consisting of sucrose octanoate and sorbitol octanoate.

13. The method of claim 12 wherein said fungi are selected from the group consisting of Pythium ultimum, Rhizoctonia solani, Fusarium graminearum, Alternaria sp., Stachybotrys chartarum, Penicillium sp., Rhizopus spp., and Muco sp.
